(19) United States
(12) Patent Application Publication
(10) Pub. No.: US 2011/0110887 A1
(43) Pub. Date: May 12, 2011

(54) RECOMBINANT HERPESVIRUS OF TURKEYS ENCODING FOR INTERLEUKIN-12

(76) Inventors: Wilhelmus Gerardus Johannes Degen, Boxmeer (NL); Virgil Elisaeth Joseph Caspar Schijns, Utrecht (NL)

(21) Appl. No.: 13/000,753

(22) PCT Filed: Jun. 22, 2009
(86) PCT No.: PCT/EP2009/057746

§ 371 Date: Dec. 22, 2010

Related U.S. Application Data
(60) Provisional application No. 61/075,079, filed on Jun. 24, 2008.

(30) Foreign Application Priority Data
Jun. 23, 2008 (EP) 08158788.3
Jun. 25, 2008 (EP) 08158920.2

(51) Int. Cl. A61K 48/00; C12N 7/00

(52) U.S. Cl. 424/85.2; 435/235.1

(57) ABSTRACT

The present invention pertains to the use of a recombinant Herpesvirus of Turkeys (HVT) containing a heterologous nucleic acid sequence that encodesinter-leukin-12, to manufacture a medicament which protects a bird against Marek's disease virus and reduces its sensitivity for microbial infection.

The present invention pertains to a recombinant Herpesvirus of Turkeys (HVT) containing a heterologous nucleic acid sequence in the HVT genome.

Recombinant HVT is for example known from EP 431 668. HVT is a commonly known Marek's Disease virus (MDV) and is widely used in safe vaccines for the effective control of Marek's disease (MD) in poultry. Recombinant HVT, which is also commonly known and used for protection against Marek's disease, offers e.g. the possibility to additionally provide protection against other pathogens by introducing a heterologous gene that encodes one or more antigens of the said other pathogen(s). Also, recombinant HVT may be a HVT that comprises genes from another serotype of Marek's disease virus. Such recombinant HVT is for example described in U.S. Pat. No. 5,965,138 and is also referred to as recombinant chimeric virus or novel avian herpes virus.

From the prior art, numerous methods are known to obtain a recombinant HVT vector containing a heterologous nucleic acid sequence introduced into the HVT genome. For example, in U.S. Pat. No. 5,965,138 (the '138 patent) it is disclosed to introduce foreign DNA in a non-essential region of the unique long viral region which is part of the genome naturally occuring in a herpesvirus of turkeys. To date it is common general knowledge to operatively link the heterologous nucleic acid to a functional promoter, e.g. a promoter naturally occurring in HVT or a heterologous one, such that the heterologous DNA is actually expressed in a host. Such methods are known from the '138 patent as mentioned here-above but are also known for example from Sondermeijer et al (in: Vaccine, 1993; 11(3): 349-58), Djeraba et al (in: Journal of Virology, February 2002, p. 1062-1070) and Mohr et al (in: International Journal of Medical Microbiology 298, 2008, 115-125). In the EP patent 431 668 it is described that the heterologous nucleic acid sequence can be derived from any pathogen, preferably an avian pathogen, which after insertion into the HVT genome can be applied to induce immunity against a disease or disorder which would be induced by that pathogen. Such pathogens could for example be Infectious Bronchitis Virus (IBV), Newcastle Disease Virus (NDV), Infectious Bursal Disease Virus (IBDV), Chicken Anaemia Agent (CAA), Reo Virus, Avian Retro Virus, Fowl Adeno Virus, Turkey Rhinotracheitis Virus, Eimeria species, Salmonella species, Escherichia coli, Mycoplasma gallisepticum and synoviae, Ornithobacterium rhinotracheale, Campylobacter etc.

However, the known recombinant HVT's have a drawback. For each vaccine that, next to a protection against Marek's disease, has to provide protection against another disease or disorder, one has to insert a heterologous nucleic acid sequence of the pathogen that induces the other disease or disorder (from now on the term “disorder” will also be used to cover “disease”). This is not attractive from an economical point of view and also decreases the safety of the use of the recombinant HVT in a vaccine.

It is an object of the present invention to overcome or at least mitigate the above mentioned drawbacks. To this end, a recombinant HVT according to the preamble has been devised, wherein the heterologous nucleic acid encodes for an IL-12 protein, which is used to manufacture a medicament which protects a bird against Marek's disease virus and reduces its sensitivity for microbial infection.

To applicants surprise it appears to be possible to reduce the sensitivity of a bird for microbial infection (i.e. an infection other than an infection with Marek's disease virus for which the HVT itself already provides protection), without the need of administering antigens derived from the corresponding microorganism, by administering a HVT vector that encodes for IL-12. Indeed, it is generally known from literature that IL-12 plays a role in the immune response to combat an infection with a microbe (i.e. a microorganism such as a bacterium, a virus or parasite). It is also known that mammals which are IL-12 deficient (i.e. which cannot produce IL-12), might have a less efficient immune response when being subjected to pathogens, and that they can be brought to a normal level by directly administering exogenous IL-12. However, it now appears that in healthy birds (i.e. being not IL-12 deficient) one can make the bird less sensitive for an infection with any microorganism, without at the same time (or in any case before an actual infection with a wild-type microorganism occurs) having to administer an antigen derived from that microorganism, even when the IL-12 is not offered as such, but in the form of DNA which is to be expressed by the HVT vector. This way, a very convenient and inexpensive method of reducing the bird's sensitivity to an infection with a microorganism other than a Marek's Disease virus is obtained (it is noted that the IL-12 that is expressed might also enhance the protection against Marek's disease). Moreover, the medicament is extremely safe since it does not need to contain any additional antigenic material apart from the HVT itself, so the risk of adverse mutual interference of different antigenic combinations can be prevented completely. However if wished, the medicament may contain additional antigenic material. This additional material could for example be material derived from other Marek's disease viruses (MDV's) when wishing to obtain optimal protection against a wide range of MDV's or even from non-MDV microorganisms.

Interleukin-12 (abbreviated as IL-12 or IL12) is a commonly known cytokine, i.a. produced by antigen-presenting cells with immunoregulatory functions in the generation of T-helper cells type 1 and cytotoxic lymphocytes (Blood, Vol. 84, No 12, Dec. 15, 1994; pp 4008-2027). IL-12 is a cytokine with a heterodimeric structure, having a molecular mass of about 70 kD. It is formed by two linked chains of approximately 40 kD and 35 kD. Among various species, the homology percentage between the various IL-12 cytokines can be as low as for example 20-40% (e.g. when comparing mammalian IL-12 with avian IL-12) while preserving the functionality of the cytokine (Degen et al; The Journal of Immunology, 2004, 172: 4371-4380).

Protection in the sense of the present patent means to induce an immune response for aiding in preventing, ameliorating or treating a disease or disorder resulting from infection with the microorganism, as a result of administering (a composition containing) one ore more antigens derived from that microorganism, such as an attenuated or killed microorganism and/or a subunit thereof, or any other substance such as a metabolite of the microorganism. Reducing sensitivity for microbial infection in the sense of the present patent means evoking that the animal shows less clinical signs and/or shows a reduced colonization of the microorganism and/or shows a reduced effect of the colonization, e.g. reduced lesions, when having an infection with that microorganism.

A composition containing antigens derived from a microorganism is usually referred to as a vaccine against that microorganism, that is, if the antigens are present in an immunologically effective amount (i.e. capable of stimulating the immune system of the target animal sufficiently to at least reduce the negative effects of a challenge with the wild-type micro-organism). The antigens are typically combined with a pharmaceutically acceptable carrier such as a liquid containing water, optionally comprising immunostimulating agents (adjuvants), which upon administration to the animal provides protection against the corresponding microorganism.

In general, a medicament can be manufactured by using art-known methods that basically comprise admixing the active compound (or a composition containing the active compound) with a pharmaceutically acceptable carrier, e.g. a liquid carrier such as (optionally buffered) water or a solid carrier such as commonly used to obtain freeze-dried vaccines, or tablets, bougies or capsules. Optionally other substances such as adjuvants, stabilisers, preservatives, humectants, viscosity modifiers, fillers or other components are added depending e.g. on the intended use, required properties, or way of administration of the medicament. For oral and parenteral administration many suitable forms are known in the art. For parenteral administration in particular liquid formulations (with dissolved, emulsified or suspended antigens) are used, but also solid formulations such as implants or an intermediate form such as a solid carrier for the antigen suspended in a liquid are known. Parenteral administration and suitable (physical) forms for the used medicaments have been known for hundreds of years.

It is noted that from WO 2004/003017 it is known that IL-12 may augment microorganism-induced immune responses or vaccination-induced immune responses, based on both cellular and humoral immunity. However, according to this known use of IL-12 in a vaccine against a pathogen, one still has to add antigens (immunogenic material) derived from the pathogen against which the vaccine should provide protection. This reference remains silent about reducing the sensitivity for an infection with a pathogen using IL-12 as such (i.e. without in combination also administering antigens derived from the pathogen), let alone using a vector virus that encodes for IL-12.

It is also noted that Kincy-Cain et al (Infection and Immunity, April 1996, pp 1437-1440) have disclosed that exogenous (i.e. not produced by the subject animal self) interleukin-12 can augment the protective immune response in mice that are orally challenged with Salmonella dublin. However, this reference teaches that the IL-12 should be administered directly by using an osmotic pump implanted subcutaneously. It may be clear that this is a reliable but economically unattractive way of providing exogenous IL-12. It is noted that the reference remains silent about effects in birds. It is commonly known that the mammalian and avian immune system differs substantially and that one cannot reasonably expect that an immune effect obtained in mammals can also be obtained in avian species. Moreover, Kincy-Cain remains silent about the option to provide the exogenous IL-12 indirectly, in particular via a vector, let alone a Herpes Virus of Turkeys. Based on the disclosure of Kincy-Cain one cannot reasonably predict or expect that the use of HVT that encodes IL-12, in particular when used in healthy (i.e. non IL-12 deficient) birds, can reduce the sensitivity of these birds for microbial infection.

In an embodiment the microbial infection is a bacterial or viral infection. It appears that the present invention is particularly suitable to reduce the sensitivity for such infections, in particular Salmonella and avian influenza infection.

In another embodiment the interleukin-12 is an avian interleukin. Although non-avian interleukin-12 could be used (given the fact that interleukin-12 is defined by its functionality) it is believed that the use of avian interleukin-12 reduces the risk of an auto-immune response against the IL-12, and provides an optimal priming result.

In yet another embodiment the bird is a chicken and the interleukin is chicken interleukin.

In yet a different embodiment the medicament is for in-ovo administration or administration to day-old (day-of-hatch) animals. These types of administration have the advantage that from the moment of hatch, one can expect an improved protection (as compared to non-vaccinated animals) against Marek's disease and a reduced sensitivity to other microorganisms. Especially for broilers (which, apart from a MD-vaccine, often will not receive any additional vaccination during their life, typically 6 to 8 weeks) such post-hatch reduced sensitivity for infections with other pathogens is economically very attractive.

The invention also pertains to Recombinant Herpesvirus of Turkeys (HVT) containing a heterologous nucleic acid sequence introduced into the HVT genome, the heterologous nucleic acid encoding for interleukin-12, for use in a medicament which protects a bird against Marek's disease virus and reduces the sensitivity for microbial infection. It also pertains to the use of a recombinant Herpesvirus of Turkeys (HVT) containing a heterologous nucleic acid sequence introduced the HVT genome, the heterologous nucleic acid encoding for interleukin-12, to protect a bird against Marek's disease virus and to reduce its sensitivity for microbial infection.

The present invention has shown to be applicable for obtaining a reduced sensitivity for infections with microorganisms that vary widely in the way they infect and colonize the target animal. In particular, the advantageous effects have been explicitly embodied for bacteria, in particular the enterobacteraceae Salmonella, as well as viruses, in particular the respiratory virus avian influenza. Since these microorganisms are really totally unrelated in their way of infection, colonization and in the way they trigger an immune response, it can therefore reasonably be expected that the invention is applicable for any microorganism, in particular any bacterium belonging to the enterobacteraceae, in particular Salmonella, or any virus, in particular any respiratory virus, in particular any influenza virus, or any other micro-organism such as the ones belonging to the Rickettsia. The invention shall now be explained in more detail using the following non-limiting examples.

### MATERIALS AND METHODS

Expression and Purification of Recombinant Chicken IL-12 (recChIL-12)

A single-chain IL-12 p40-p35 heterodimer molecule was generated (for aminoacid sequences see Degen et al. in The Journal of Immunology, 2004, 172: 4371-4380), in which the p40 chain was linked to the p35 chain by an in-frame (G4S)3-linker; this molecule was designated ChFlexi-IL-12 as described in Degen et al. This single-chain construct was expressed in HVT using the methods as described in EP 0 431 668.

Manufacturing a HVT-IL-12 Virus and a Medicament Containing that Virus

HVT having inserted chicken IL-12 was made using the methods as described in EP 0 431 668 with the IL-12 DNA as known from Degen et al. (The Journal of Immunology, 2004, 172: 4371-4380). The recombinant virus was suspended in phosphate buffered saline (also called “PBS” or simply “saline”) at 30.000 pfu/ml to constitute a medicament.

In vitro Bioactivity of recChIL-12: Proliferation and Induction of ChIFN-γ Synthesis by Chicken Spleen Cells

Chicken primary splenocytes were seeded in triplicate in a 96-well plate at a density of 0.5×10⁶ cells/well in 100 μl and incubated with the indicated amounts of either recChIL-12 or heat-inactivated recChIL-12 (10 min. at 100° C.). Forty-eight hours after the addition of proteins, proliferation of cells was assessed after 18-20 hrs [methyl-³H]-thymidine (18.5 kBq at 25 μl/well) uptake. The incorporated radioactivity was counted using an LKB beta counter (LKB Instruments, Gaithersburg, Md.). ChIL-12 activated splenocytes also showed increased production of ChIFN-γ (see Degen et al; The Journal of Immunology, 2004, 172: 4371-4380).

HVT-Ch IL-12 Bioactivity

When compared to the supernatants of cells infected by empty HVT vector virus and mock infected cells, the supernatants of HVT-ChIL-12 infected cells showed clear evidence of ChIL-12 bioactivity as measured by increased proliferation of freshly isolated splenocytes.

Administration of Cytokine Expressing HVT

One (1) day-old female non IL-12 deficient SPF chickens (20 animals per group) were i.m. injected in the leg muscle with 0.1 ml of the medicament as described here-above. Control animals were administered either phosphate buffered saline containing HVT-PB1 as an empty control vector (at a dose of 3000 pfu/animal), or just the saline. It is noted that instead of the i.m. (intramuscular) injection of the chickens one could also use in-ovo administration given the fact that it is commonly known that vaccination against Marek's disease with HVT can successfully be obtained by in-ovo administration.

Salmonella Infection and Resistance Assessment

One day before challenge infection the Salmonella enteritidis (SE) (Salmonella enterica subsp. enterica serovar Entertidis) strain was freshly cultured overnight on sheep blood agar medium from a freeze-dried stock preparation by routine procedures. Challenge inocula were prepared in sterile meat extract broth and diluted appropriately after cell counting. The viable cell concentration of the inoculum was confirmed by colony counts on sheep blood agar. At 7 days of age (6 days post injection of HVT construct) animals were orally infected by gavage with 0.5 ml, containing 10⁶ cfu Salmonella/ml.

For analysis of resistance to Salmonella infection cloaca swabs were taken at 3, 7, 11 and 15 days after infection. In addition, animals were sacrificed at day 15 for monitoring of salmonella colonization in liver, spleen and coecal content. Colonization (cfu quantitation) in solid organs (liver and spleen) was determined by sampling as described below.

The surface of liver, spleen and cecum was disinfected locally with a hot spatula. The cecum and the cloaca were sampled using a swab. This swab was inoculated directly on Brilliant Green Agar modified +60 μg/ml nalidixic acid (BGAm+nal) and again after 16-24 hours enrichment at 37° C. in 9 ml buffered peptone water containing 60 μg/ml nalidixic acid (BPW+nal). The liver and spleen were inoculated on BGAm+nal using a sterile disposable inoculation loop. The organs were dipped in boiling water, crushed in a stomacher in addition of 9 ml BPW+nal and after 16-24 enrichment at 37° C. cultured on BGAm+nal. Cloaca swabs were inoculated directly on BGAm and again after 16-24 hours enrichment at 37° C. in 9 ml BPW+nal. After 16-24 hours inoculation at 37° C., all BGAm plates were screened for the presence of colonies that were suspected to be Salmonella enteritidis (red colonies). Suspicious colonies were agglutinated with D₁-antiserum (Difco Laboratories, Detroit, Mich., anti-O 1, 9 and 12). The reisolation of the challenge strain was scored semi-quantitively in the following way:

Number of SE colonies per plate after direct inoculation:

>100=6, 11-100=5, 1-10=4.

Number of SE colonies per plate after enrichment:

>100=3, 11-100=2, 1-10=1.

Avian Influenza Infection and Resistance Assessment

Avian influenza A subtype H9N2 virus (isolate A/Chicken/United Arab Emirates/99) was produced in eggs using routine procedures. The aqueous phase containing the virus suspension was diluted in 0.01 M phosphate buffered saline and used as challenge material. At 14 days of age (13 days post injection of the HVT construct) the birds were challenged with 10E8.8 EID50/ml H9N2 virus via the spray route. For the spray challenge 10 ml of H9N2 virus (10E8.8 EID50/ml) was mixed with an equal volume of saline and the resulting 20 ml of virus suspension was sprayed into the isolator (isolator volume: 0.79 m³), using an airbrush with compressor (1.5 atm; average droplet size 50 μm), with closed air circulation. The air circulation was re-opened after 10 minutes.

For analysis of resistance to avian influenza the birds were monitored for clinical respiratory signs and scored at day 7 post challenge. Birds that had severe (visible) difficulties with breathing were scored positive, the others were scored negative.

### RESULTS

In vitro Bioactivity of Cytokine-expressing Recombinant HVT

Before the start of each animal experiment all batches of HVT-ChIL-12 were tested in vitro for bioactivity, using one of the hallmarks of IL-12 activity; i.e. proliferation of freshly-isolated splenocytes as reported for mammals and recently reported for chickens (Degen et al; The Journal of Immunology, 2004, 172: 4371-4380)

Post-hatch Resistance to Salmonella Infection

The effect of HVT-ChIL-12 to reduce the sensitivity for infection was compared with the empty control vector HVT-PB1. For each group the kinetics of Salmonella infection was determined as reflected by cfu levels found in cloaca swabs at several time points post-infection. As shown in FIG. 1, HVT-ChIL-12, conferred significantly (P<0.05) reduced colonization (approximately 10-100-fold), when compared to responses evoked by the empty HVT vector (PB1) or saline group.

FIG. 2 shows that HVT-ChIL-12 significantly (P<0.05) reduced colonization in livers (FIG. 2A) and coecal tonsils (FIG. 2B).

Post-hatch Resistance to Avian Influenza Infection

The effect of HVT-ChIL-12 to reduce the sensitivity for infection was compared with the empty control vector HVT-PB1. As shown in FIG. 3, the clinical respiratory signs after challenge were significantly reduced by the administration of the HVT-ChIL-12, when compared to responses evoked by the empty HVT vector (PB1) or saline group.

### DESCRIPTION OF THE FIGURES

FIG. 1. Effect of HVT-ChL-12 on cloaca swab scores during Salmonella enterititis infection. Cfu counts in cloaca swabs at different time points after infection expressed as means±SD.

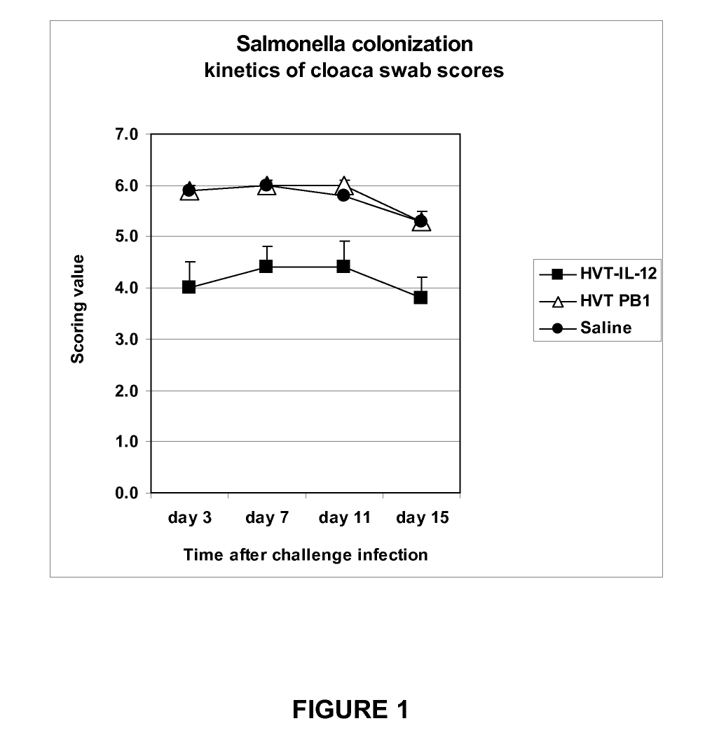

FIG. 2. Effect of HVT-ChL-12 on colonization during Salmonella enterititis infection. Colonization levels of liver (A) and coeca (B) at day 15 post infection are shown.

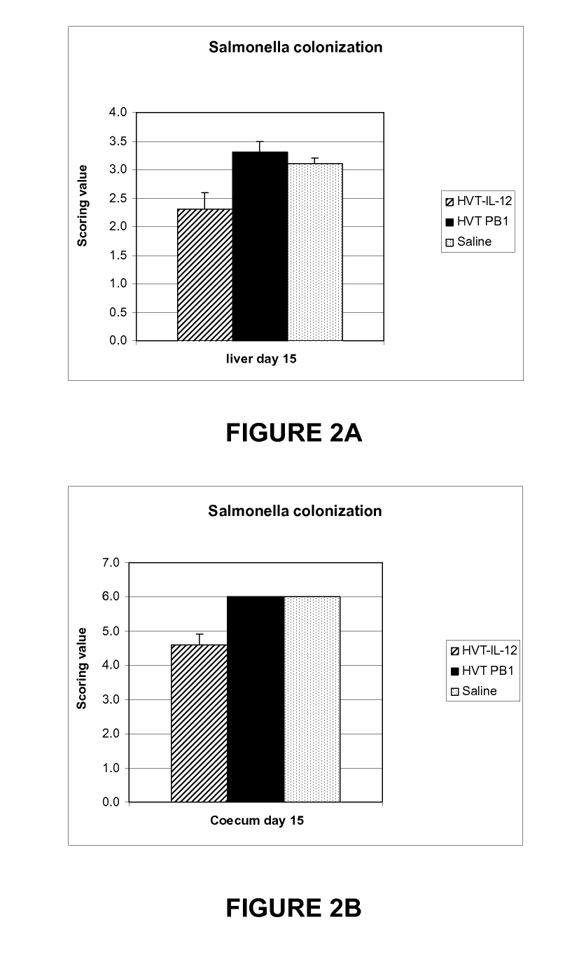

FIG. 3 Effect of HVT-ChL-12 on clinical signs during avian influenza infection. Positive clinical respiratory signs at day 7 are shown.

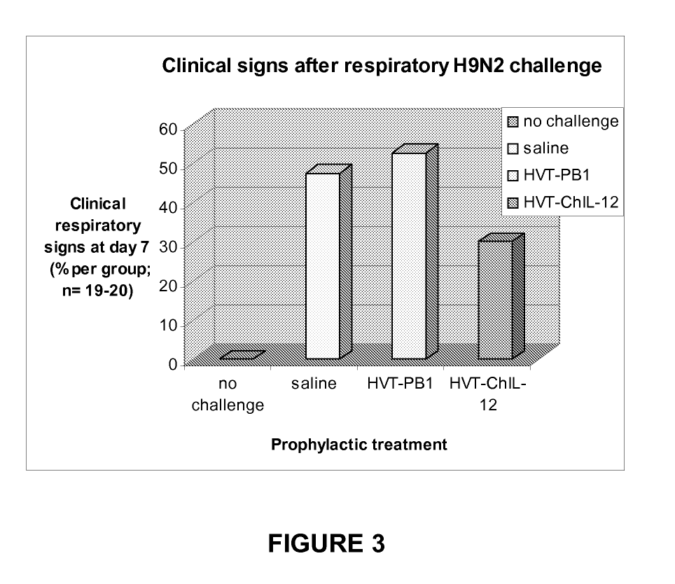

### CONCLUSION

Together these observations show that IL-12, when expressed by a HVT, acts as a safe host-derived reducer of a bird's sensitivity to microbial infection. The data make clear that HVT-expressed IL-12 may act as a substitute for the preventive use of antibiotics or as a substitute for vaccination against infections. Overall, using the novel construct a medicament can be made for significantly reducing a bird's sensitivity to microbial infections in general without the need of administering antigens of the corresponding microorganisms before an actual infection with these microorganisms takes place. Since the novel construct is based on HVT, it inherently provides protection against Marek's disease. If the purpose of the use of the novel construct, in addition to the general reduction in sensitivity to microbial infection, is protection against Marek's disease, the medicament may additionally comprise antigens from other serotypes of MDV, such as live attenuated MDV serotype 1, to provide superior protection against MD. As known from U.S. Pat. No. 5,965,138, these antigens from other serotypes of MDV may also form part of the recombinant HVT itself.

1. A method of protecting a bird against Marek's disease virus comprising administering in-ovo or to animals a recombinant Herpesvirus of Turkeys (HVT) containing a heterologous nucleic acid sequence that encodes interleukin-12; wherein said administering reduces the bird's sensitivity for a microbial infection.

2. The method of claim 1, characterised in that the microbial infection is a bacterial or viral infection.

3. The method of claim 2, characterised in that the bacterial infection is a Salmonella infection or the viral infection is an avian influenza infection.

4. The method of claim 1, characterised in that the interleukin-12 is an avian interleukin.

5. The method of claim 4, characterized in that the bird is a chicken and the interleukin is chicken interleukin.

6. The method of claim 1, characterised in that the administration is to day-old animals.

7. A Recombinant Herpesvirus of Turkeys (HVT) that comprises a heterologous nucleic acid sequence that encodes interleukin-12, wherein said HVT is for use in a medicament which protects a bird against Marek's disease virus and that reduces its sensitivity for microbial infection.

8. A method of reducing a bird's sensitivity for microbial infection comprising administering in-ovo or to animals a recombinant Herpesvirus of Turkeys (HVT) containing a heterologous nucleic acid sequence that encodes interleukin-12.

9. The method of claim 8 characterised in that the administration is to day-old animals.
